(19) United States
(12) Patent Application Publication
(10) Pub. No.: US 2017/0372004 A1
(43) Pub. Date: Dec. 28, 2017

(54) METHODS FOR CLASSIFYING ORGANISMS BASED ON DNA OR PROTEIN SEQUENCES

(71) Applicant: AgBiome, Inc., Research Triangle Park, NC (US)

(72) Inventors: James R. Henriksen, Cary, NC (US); Daniel J. Tomso, Bahama, NC (US)

(21) Appl. No.: 15/535,757

(22) PCT Filed: Dec. 17, 2015
(86) PCT No.: PCT/US2015/066446

§ 371 Date: Jun. 14, 2017

Related U.S. Application Data
(60) Provisional application No. 62/095,658, filed on Dec. 22, 2014.

(51) Int. Cl. G06F 19/22; C12Q 1/68

(52) U.S. Cl. CPC G06F 19/22; C12Q 1/68

(57) ABSTRACT

Methods for classifying organisms using whole or partial genome sequence data are provided. The methods use DNA or protein sequence data and clustering algorithms to assign organisms a protein morphotype (‘pmorph’) based on a given clustering parameter. Pmorphs can be generated quickly from whole genome sequence data, even at draft sequence quality levels, and can resolve fine differences between related organisms. In this manner, organisms can be classified and duplicate organisms within a collection can be identified.

### CROSS REFERENCE TO RELATED APPLICATION

This application claims the benefit of U.S. Provisional Application Serial No. 62/095,658, filed Dec. 22, 2014; the contents of U.S. Provisional Application Serial No. 62/095,658 are herein incorporated by reference in their entirety.

### FIELD OF THE INVENTION

The invention is drawn to methods of classifying organisms.

### BACKGROUND OF THE INVENTION

Prokaryotic cultures are composed of populations of individual strains or isolates of bacterial species. They may be composed of nearly clonal, closely related, or mixed cultures. Unambiguous identification and classification of related bacterial isolates, however, is challenging (Mende et al. (2013) Nature Methods 10(9): 881-84). Accurate identification and classification of new or existing bacterial cultures isolates requires robust methods of identification. Proper identification and classification of bacterial isolates has broad utility in diverse fields ranging from human health to agriculture.

Individual bacteria are microscopic and difficult to examine and bacterial cultures, including colonies, often have similar appearances. Conventional microbiological practice has relied on physiological characterization of bacteria. For example, cultures can be grown on diverse media using different carbon sources, selective agents, or colorimetric indicators. All of these methods rely on the differential facility of the microbe in question to grow under the specific culture conditions selected. These methods are limited, however, in that they require an initial understanding of specific physiological conditions that are discriminatory between microbes and the proper conditions to express such differences. In addition, many important physiological differences can be difficult, costly or imposable to measure. They are therefore inherently weak or useless for classifying new isolates. Additionally, otherwise unrelated microbes may display some similar physiological characteristics, which can lead to misidentification and misclassification. Finally, these approaches are often not applicable to mixed cultures, organisms that are not available, or organisms that are not culturable in isolation or in sufficient number to carry out physiological tests. In the current state of the art, large amounts of genome sequence information can be determined for even a single cell. Ishoey, et al. (2008 11: 3: 198-204.

DNA sequence based methods have improved microbial classification to some extent. In particular, sequence comparisons of the DNA that encodes the 16s ribosomal subunit (16s rDNA) have been used extensively to identify and classify bacteria. 16s rDNA classification is the general standard used to resolve microbes at the species level. However, 16s rDNA is insufficiently specific to resolve individual strains of microbes, even though such microbes may be substantially different from each other in terms of function, physiology, and evolutionary history. There are large amounts of important diversity within isolates that have even identical rRNA genes. In other words, 16s rDNA classification will incorrectly group bacteria that are different in meaningful ways at the genome and functional level. For example, strains of Bacillus anthracis, Bacillus cereus, and Bacillus thuringiensis have 16S genes that differed by one or zero nucleotides, and Bacillus mycoides by less than four, despite the radically different characteristics and pathogenicity of these strains. Other DNA-based methods have had limited success in resolving microbes beyond the species level. Multi-locus sequence typing (MLST), in which many sequenced genes are compared, is sometimes used, but generally requires specific foreknowledge of the genome compositions of a group of microbes. Furthermore, MLST is most effective with closely-related microbes, and many of the DNA amplification methods associated with recovering specific loci fail to work on diverse uncharacterized bacterial isolates. Concatenated core gene trees of all shared genes are more robust over large phylogenomic distances, but are sensitive to ortholog/paralog problems, inter- and intra-gene recombination, and horizontal gene transfer. These methods are also, in general, subject to severe computational and organizational bottlenecks that to date prevent reasonable deployment for microbial identification work.

Whole genome sequence data in principal should allow unambiguous identification and classification of bacteria. However, current methods for whole-genome comparison are slow and unable to scale across large (tens to hundreds of isolates) microbial collections. For example, large-segment genome sequence alignment can readily distinguish two microbes, but when systematically applied to large numbers of multiple genomes, such methods are subject to severe computational and organizational bottlenecks that to date prevent reasonable deployment for microbial identification work.

In silico DNA-DNA hybridization can yield metrics such as average nucleotide identity (Kim, Marakeby 2014, US 20140258299), but they do not capture functional information and suffer from severe computational and organizational bottlenecks that to date prevent reasonable deployment for microbial identification work.

### SUMMARY OF THE INVENTION

Methods for classifying organisms using whole or partial genome sequence data are provided. The methods use coding DNA or protein sequence data and clustering algorithms to assign organisms a protein morphotype (‘pmorph’) based on a given clustering parameter representing shared sequences. Pmorphs can be generated quickly from whole genome sequence data, even at draft sequence quality levels, and can resolve fine differences between related organisms. In this manner, organisms can be classified and duplicate organisms within a collection can be identified.

### BRIEF DESCRIPTION OF THE DRAWINGS

FIG. 1 depicts a heatmap of shared protein clusters for genomes of 5,650 microbial isolates. Isolates are represented by position along the horizontal and vertical axes, and the distance between each pair of isolates is represented by the color (ranging from 0 indicating no differing protein clusters, to 1, indicating no shared protein clusters).

FIG. 2 depicts a histogram of the number of isolates in distinct pmorph classification groups in a set of 176 genomes of microbial isolates that include multiple genome sequences of the same clonal isolate.

FIG. 3 depicts a heatmap of shared protein clusters for 176 genomes of microbial isolates that include multiple genome sequences of the same clonal isolate. Isolates are represented by position along the horizontal and vertical axes, and the distance between each pair of isolates is represented by the color (ranging from 0 indicating no differing protein clusters, to 1, indicating no shared protein clusters).

FIG. 4 depicts a network clustering diagram of 176 genomes of microbial isolates that include multiple genome sequences of the same clonal isolate. Circles indicate isolate genomes, pmorph groups are clustered together and are the same colored, and distance within group is proportional with number of shared proteins.

### DETAILED DESCRIPTION OF THE INVENTION

Methods for typing bacterial isolates using whole or partial genome sequence data are provided. The methods use a set of robust steps that have been optimized to allow accurate and scalable identification and classification of known and newly-discovered organisms. In certain embodiments, and as described in detail herein, at least some of the steps of the method may be performed using a computer. The method uses sequence data and clustering algorithms to assign organisms a protein morphotype (‘pmorph’). Pmorphs can be generated quickly from whole genome sequence data, even at draft sequence quality levels, and can resolve fine differences between microbes. Pmorph classification can segregate microbes that appear identical by 16s rDNA typing. The methods can be scaled to large numbers of diverse isolates and to physiologically relevant characteristics. This method, properly parameterized, can be robust to sequencing errors, ortholog/paralog problems, inter- and intra- gene recombination, horizontal gene transfer (HGT), and chimeric evolutionary histories. Widespread HGT and even chimeric origins of large phylogenetic groups are common and important (Nelson-Sathi S et al. (2012) PNAS, 109:20537-20542). Unlike other methods, this method captures this type of non-hieratical gene transfers.

### I. Sequence Information Used for Classification

The methods provided herein classify organisms using a bioinformatics approach to determine relationships between organisms based on DNA sequence information obtained from those organisms. “Classification”, “classify”, and “classifying” refer to grouping of organisms in relationship to other organisms. For example, organisms can be classified in traditional hierarchical or taxonomic systems such as Genus and species classification. Organisms can also be classified accordingly to known phylogenetic parameters, evolutionary distance, functional criteria, physiological characteristics, or any other parameter useful for differentiating organisms. In some embodiments, organisms can be classified based on numbers of shared genes or proteins, or based on the sequence identity of the same protein or gene encoding the same protein, as disclosed elsewhere herein.

Sequence information used for classification can either be nucleic acid or amino acid sequence information. In certain embodiments, the method comprises selection of certain sequences for comparison. In some embodiments, whole or partial genomic DNA sequence information is used for classification. Whole or partial genomic DNA sequence information can be obtained as a step in the method, or can be obtained from public or private databases. In some embodiments, the whole or partial genomic DNA of bacterial isolates is sequenced and used for classifying the isolates according to the methods provided herein. Any method of genome sequencing can be used to obtain whole or partial genomic DNA sequence information, including but not limited to shotgun sequencing, Sanger sequencing, next generation sequencing, or single molecule sequencing, among others. Open reading frames (ORFs) can be predicted or identified from the whole or partial genomic sequence information. ORFs or genes can be identified using common genome sequence analysis software available in the art. For example, the getorf program, included in the EMBOSS software package (Rice, P. et al. (2000) Trends in Genetics 16(6):276-277), versions 6.3.1 available from EMBnet at EMBOSS web sites, among other sources) determines ORFs from sequences, and the Prodigal program Hyatt D. et al. (2010) BMC Bioinformatics 11(1):119, version 2.6.0 and later is available from the Oak Ridge National Laboratory web site, among other sources). The identified ORFs can be used themselves for classification of the organism or predicted expressed amino acid sequences from the identified ORFs can be used for classification of the organism. Alternatively, if the predicted expressed protein sequences of an organism or group of organisms is known, those predicted expressed protein sequences can be used in the methods of classifying disclosed herein.

In some embodiments, all ORFs above a certain size limit are identified and used directly for classification of the source organism, or used for protein prediction, wherein the predicted proteins and used for classification of the source organism. In certain embodiments, the method comprises selection of the size of the sequences for comparison. For example, ORFs having at least 12, 15, 18, 21, 24, 27, 30, 45, 60, 75, 90, 115, 130, 150, 180, 210, 240, 270, 300, 450, 600, 750, 900, 1000, 1500, 1750, 2000, 3000, 4000, 5000, 10,000, 25,000 or more nucleic acids are identified and used for classification of the source organism or for protein prediction. In other embodiments, groups of ORFs identified by upstream sequences involved in gene expression can be used for classification or protein prediction. Alternatively, groups of ORFs with homology to genes of interest or conserved gene regions can be used for classification or protein prediction according to the methods disclosed herein.

The various methods provided herein allow for the use of a partial genome of a given organism. In specific methods, at least 10%, 20%, 30%, 40%, 50%, 60%, 70%, 80%, 90%, or 99% of a given organism's genomic DNA is used in the methods described herein. Alternatively, when open reading frames are used, at least 10%, 20%, 30%, 40%, 50%, 60%, 70%, 80%, 90%, or 99% of the OFRs from a given organism is used in the methods.

Any organism can be classified by the methods disclosed herein. Thus, the methods and systems of the invention can provide a meaningful classification of an organism of interest based on a particular criterion of interest (e.g., sequence region, pathogenicity, pesticide resistance). For example, prokaryotic organisms such as bacteria or archea strains can be classified by the methods disclosed herein. Alternatively, eukaryotic organisms or viruses can be classified by the methods disclosed herein. Whole or partial genomic DNA sequence information obtain from the organisms to be classified can be obtained by whole genome sequencing or partial genome sequencing of a purified population of the organism, or by sequencing of a complex sample. By “complex sample” is intended any sample having DNA from more than one species of organism. In specific embodiments, the complex sample is an environmental sample, a biological sample, or a metagenomic sample. As used herein, the term “metagenome” or “metagenomic” refers to the collective genomes of all microorganisms present in a given habitat (Handelsman et al., (1998) Chem. Biol. 5: R245-R249; Microbial Metagenomics, Metatranscriptomics, and Metaproteomics. Methods in Enzymology vol. 531 DeLong, ed. (2013)). Environmental samples can be from soil, rivers, ponds, lakes, industrial wastewater, seawater, forests, agricultural lands on which crops are growing or have grown, or any other source having biodiversity. In an embodiment, there is no particular upper limit on the number of organisms that can be classified using the methods and systems of the invention. Thus, the methods allow for the classification of at least 2, 5, 10, 50, 100, 200, 500, 1000, 10,000, 100,000, 1×10⁶, 1×10⁷, 1×10⁸, ×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰ or more organisms.

Complex samples also include colonies or cultures of microorganisms that are grown, collected in bulk, and pooled for storage and DNA preparation. In certain embodiments, complex samples are selected based on expected biodiversity that will allow for classification of a variety of organisms. In some embodiments, the methods described herein are used to reduce redundancies in a collection of microorganisms by classifying the organisms in the collection and determining of individual organisms are replicated within the collection.

### II. Generating Distance Measurements

The various methods of classifying organism provided herein generate a distance measurement between at least two organisms. As used herein, a distance measurement means an estimate of the genetic relatedness or two organisms which varies on a discrete or continuous scale, wherein a larger distance indicates less relatedness, and a smaller distance indicates more relatedness. In specific embodiments, the DNA or predicted amino acid sequences can be compared using distance metrics. In certain embodiments, the method comprises selection of the method used for detemination of the distance metric. These distance metrics can include either or any combination of Euclidean distance, Sorensen distance, Jaccard distance, Pearson correlation, vector distances, Chi square, city block, or ordination methods that optionally comprise use of Principal Component Analysis (PCA), Bray-Curtis ordination or Bray-Curtis dissimilarity, and nonmetric multidimensional scaling (NMS or NMDS). Such distance measurements are set forth in, for example, Dinsdale, et al. (2013) Statistical Genetics and Methodology 4:41 and Beals, E.W. (1984) Adv. Ecol. Res. 14: 21-29, both of which are herein incorporated by reference in their entirety. See, also, Legendre P. and L. Legendre (1998) Numerical Ecology Amsterdam: Elsevier Science: 853 and McCune, B. and J. B. Grace (2002) Analysis of Ecological Communities. MjM Software Design: Gleneden Beach, Oregon: 300, and Ramette, Alban (2007) “Multivariate Analyses in Microbial Ecology.” FEMS Microbiology Ecology 62, no. 2: 142-60; each of which is herein incorporated by reference in their entirety.

Continuous distance metrics should output a numerical value of distance or similarity between the organisms being compared. For discrete metrics, an appropriate threshold value can be applied to convert continuous distance measurements into discrete values.

### III. Clustering

The methods disclosed herein employ a clustering approach to group similar or identical organisms as a means of classification. As used herein, “clustering” refers to a method of grouping organisms based on a chosen characteristic, or clustering parameter. In certain embodiments, the method comprises selection of clustering method and/or parameters used. Clustering analysis is useful for grouping objects into categories based on their dissimilarities or similarities and works well when there are discontinuities in the samples. For example, organisms can be clustered based on clustering parameters such as the number of shared genes or proteins, the percent sequence identity of the same gene or protein, functional characteristics, metabolic and physiological characteristics, evolutionary distance, or any given relative or absolute parameter that can be determined for each organism.

In specific embodiments, the clustering parameter employed comprises the percent of shared proteins that have a homology cutoff (98.5% sequence similarity as implemented by CD-HIT) with the DBSCAN algorithm (with parameters of 98.5% shared proteins distance and a minimum sample of 1). DB SCAN algorithm is described in “A Density-Based Algorithm for Discovering Clusters in Large Spatial Databases with Noise” Ester, M., H. P. Kriegel, J. Sander, and X. Xu, (1996) In Proceedings of the 2nd International Conference on Knowledge Discovery and Data Mining, Portland, Oreg., AAAI Press, pp. 226-231, herein incorporated by reference in its entirety. Other algorithms that could also be employed include, but are not limited to, MeanShift as described in “Mean shift: A robust approach toward feature space analysis.” D. Comaniciu and P. Meer (2002) IEEE Transactions on Pattern Analysis and Machine Intelligence; and Affinity Propagation as described in Brendan J. Frey and Delbert Dueck (2007) “Clustering by passing messages between data points” Science 315 (5814): 972-976, doi:10.1126/science.1136800. PMID 17218491; both of which are incorporated by reference in their entirety.

Other algorithms useful for clustering include, but are not limited to, K-means clustering, Classification Trees, Random Forests, Multidimensional Scaling, Linear Discriminant Analysis, Principal Component Analysis, and Canonical Discriminant Analysis. See, Dinsdale, et al. (2013) Statistical Genetics and Methodology 4: 41, herein incorporated in the entirety. The methods of clustering can use hierarchical clustering, identification of connected components, connectivity-based clustering, distribution-based clustering, density-based clustering, single-linkage clustering, Marcov clustering (MCL), and/or centroid clustering among others. Thus, in some embodiments, the methods disclosed herein can be performed on a computer.

Comparing sequences for clustering analysis can be performed by sequence alignment or any other method specific for the clustering parameter chosen. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. The BLAST programs of Altschul et al (1990) J Mol. Biol. 215:403 are based on the algorithm of Karlin and Altschul (1990) supra. Alignment may also be performed manually by inspection. The CD-HIT program, available from the weizhongli-lab.org server implements an algorithm that significantly accelerates clustering of homologous sequences by using short word filtering and a greedy incremental clustering algorithm citation. See, for example, Limin Fu, Beifang Niu, Zhengwei Zhu, Sitao Wu and Weizhong Li (2012), CD-HIT: accelerated for clustering the next generation sequencing data. Bioinformatics 28 (23): 3150-3152 and Weizhong Li & Adam Godzik (2006) Bioinformatics 22:1658-9, each of which are herein incorporated by reference. See, also, Weizhong Li, et al., (2001) Bioinformatics 17:282-283 and Weizhong Li, et al. (2002) Bioinformatics 18: 77-82.

Predicted protein sequences from Prodigal gene calls (Hyatt et al., “Prodigal: Prokaryotic Gene Recognition and Translation Initiation Site Identification,” BMC Bioinformatics 11, no. 1 (Mar. 8, 2010): 119. doi:10.1186/1471-2105-11-119) of assembled genome sequences of microbial isolates can be generated. These amino acid sequences can be clustered at cutoffs of 98.5% amino acid identity using the CD-HIT program. A matrix of shared protein clusters above this cutoff can then be constructed for all pairwise comparisons of isolates, and normalized to the average genome size of the pair. This normalized matrix can be clustered using the DBSCAN algorithm with a neighborhood size corresponding to 98.5% shared proteins. The preceding steps can be implemented in a python script using the numpy, scipy, and sklearn numerical libraries. In other embodiments, the neighborhood size of shared proteins can be influenced by quality of gene calls, length of gene calls, and size of the data set. In specific embodiments, at least 75%, 80%, 85%, 90%, 91%, 92%, 93%, 94%, 95%, 96%, 97%, 97.5%, 98%, 98.5%, 99%, 99.5% or greater of the gene calls, shared proteins or open reading frames are employed in the clustering analysis.

The clustering parameter for classification can be the sequence similarity between the same proteins coded for in the genomes of the organisms being classified. For example, in order to cluster organisms based on the protein-specific sequence similarity, the percent sequence similarity can be determined between the same proteins coded for in each organism. In certain embodiments, the percent of sequence similarity is determined for every protein of each genome in the organisms being classified. In other embodiments, the percent of sequence similarity is determined for at least 25, at least 50, at least 60, at least 70, at least 80, at least 90, at least 100, at least 125, at least 150, at least 175, at least 200, at least 250, at least 300 or more predicted genes or proteins in a organisms genome. Using the percent sequence similarity, protein similarity groups can be generated at protein cutoffs with CD-HIT or with tools such as ITEP, getHomolog, or LSBR that can group sequences with more distant homology or more complex evolutionary history. In embodiments in which cutoffs are used, groups can be assigned to those sequences (DNA or amino acid) that have distances below the cutoff. If an individual sequence does not have a score, the sequence would form a gene presence/absence matrix of the genome. Thus, the number of present and/or absent sequences relative to at least two organisms can constitute a clustering parameter. Protein-group specific sequence divergence criteria or overall distances can be used to normalize values for clustering in some embodiments.

Alternatively, the clustering parameter can be the number of shared similar genes or proteins. For example, in order to cluster organisms based on the number of shared similar proteins, a cutoff value for similar proteins must be established. That is, proteins having at least 90%, at least 91%, at least 92%, at least 93%, at least 94%, at least 95%, at least 95.5%, at least 96%, at least 96.5%, at least 97%, at least 97.5%, at least 98%, at least 98.2%, at least 98.5%, at least 98.7%, at least 99%, at least 99.2%, at least 99.5%, at least 99.7%, or at least 99.9% sequence identity can be classified as similar proteins. Further, protein similarity can be determined over any length of amino acid sequence sufficient to ascertain protein similarity. For example, protein similarity can be determined over a length of at least 10, at least 15, at least 20, at least 21, at least 22, at least 23, at least 24, at least 25, at least 26, at least 27, at least 28, at least 29, at least 30, at least 32, at least 34, at least 36, at least 38, at least 40, at least 45, at least 50, at least 55, or at least 60 amino acids.

In specific embodiments, the clustering parameters employed are those set forth in Table 1 and Table 2.

TABLE 1
Non-limiting variations of clustering parameters

| % shared | |
|---|---|
| proteins | |
| between | |
| organisms | % sequence identity between two proteins |
| 90% | 90%, 91%, 92%, 93%, 94%, 95%, 95.5% 96%, 96.5%, |
| | 97%, 97.5%, 98%, 98.5% or 99%, or 99.5% |
| 91% | 90%, 91%, 92%, 93%, 94%, 95%, 95.5% 96%, 96.5%, |
| | 97%, 97.5%, 98%, 98.5% or 99%, or 99.5% |
| 92% | 90%, 91%, 92%, 93%, 94%, 95%, 95.5% 96%, 96.5%, |
| | 97%, 97.5%, 98%, 98.5% or 99%, or 99.5% |
| 93% | 90%, 91%, 92%, 93%, 94%, 95%, 95.5% 96%, 96.5%, |
| | 97%, 97.5%, 98%, 98.5% or 99%, or 99.5% |
| 94% | 90%, 91%, 92%, 93%, 94%, 95%, 95.5% 96%, 96.5%, |
| | 97%, 97.5%, 98%, 98.5% or 99%, or 99.5% |
| 95% | 90%, 91%, 92%, 93%, 94%, 95%, 95.5% 96%, 96.5%, |
| | 97%, 97.5%, 98%, 98.5% or 99%, or 99.5% |
| 95.5% | 90%, 91%, 92%, 93%, 94%, 95%, 95.5% 96%, 96.5%, |
| | 97%, 97.5%, 98%, 98.5% or 99%, or 99.5% |
| 96% | 90%, 91%, 92%, 93%, 94%, 95%, 95.5% 96%, 96.5%, |
| | 97%, 97.5%, 98%, 98.5% or 99%, or 99.5% |
| 96.5% | 90%, 91%, 92%, 93%, 94%, 95%, 95.5% 96%, 96.5%, |
| | 97%, 97.5%, 98%, 98.5% or 99%, or 99.5% |
| 97% | 90%, 91%, 92%, 93%, 94%, 95%, 95.5% 96%, 96.5%, |
| | 97%, 97.5%, 98%, 98.5% or 99%, or 99.5% |
| 97.5% | 90%, 91%, 92%, 93%, 94%, 95%, 95.5% 96%, 96.5%, |
| | 97%, 97.5%, 98%, 98.5% or 99%, or 99.5% |
| 98% | 90%, 91%, 92%, 93%, 94%, 95%, 95.5% 96%, 96.5%, |
| | 97%, 97.5%, 98%, 98.5% or 99%, or 99.5% |
| 98.5% | 90%, 91%, 92%, 93%, 94%, 95%, 95.5% 96%, 96.5%, |
| | 97%, 97.5%, 98%, 98.5% or 99%, or 99.5% |
| 99.5% | 90%, 91%, 92%, 93%, 94%, 95%, 95.5% 96%, 96.5%, |
| | 97%, 97.5%, 98%, 98.5% or 99%, or 99.5% |
| 100% | 90%, 91%, 92%, 93%, 94%, 95%, 95.5% 96%, 96.5%, |
| | 97%, 97.5%, 98%, 98.5% or 99%, or 99.5% |

TABLE 2
Non-limiting variations of clustering parameters

| % sequence | |
|---|---|
| identity | |
| between 2 | |
| proteins | % shared proteins between the organisms |
| 90% | 90%, 91%, 92%, 93%, 94%, 95%, 95.5% 96%, 96.5%, |
| | 97%, 97.5%, 98%, 98.5% or 99%, or 99.5% |
| 91% | 90%, 91%, 92%, 93%, 94%, 95%, 95.5% 96%, 96.5%, |
| | 97%, 97.5%, 98%, 98.5% or 99%, or 99.5% |
| 92% | 90%, 91%, 92%, 93%, 94%, 95%, 95.5% 96%, 96.5%, |
| | 97%, 97.5%, 98%, 98.5% or 99%, or 99.5% |
| 93% | 90%, 91%, 92%, 93%, 94%, 95%, 95.5% 96%, 96.5%, |
| | 97%, 97.5%, 98%, 98.5% or 99%, or 99.5% |
| 94% | 90%, 91%, 92%, 93%, 94%, 95%, 95.5% 96%, 96.5%, |
| | 97%, 97.5%, 98%, 98.5% or 99%, or 99.5% |
| 95% | 90%, 91%, 92%, 93%, 94%, 95%, 95.5% 96%, 96.5%, |
| | 97%, 97.5%, 98%, 98.5% or 99%, or 99.5% |
| 95.5% | 90%, 91%, 92%, 93%, 94%, 95%, 95.5% 96%, 96.5%, |
| | 97%, 97.5%, 98%, 98.5% or 99%, or 99.5% |
| 96% | 90%, 91%, 92%, 93%, 94%, 95%, 95.5% 96%, 96.5%, |
| | 97%, 97.5%, 98%, 98.5% or 99%, or 99.5% |
| 96.5% | 90%, 91%, 92%, 93%, 94%, 95%, 95.5% 96%, 96.5%, |
| | 97%, 97.5%, 98%, 98.5% or 99%, or 99.5% |
| 97% | 90%, 91%, 92%, 93%, 94%, 95%, 95.5% 96%, 96.5%, |
| | 97%, 97.5%, 98%, 98.5% or 99%, or 99.5% |
| 97.5% | 90%, 91%, 92%, 93%, 94%, 95%, 95.5% 96%, 96.5%, |
| | 97%, 97.5%, 98%, 98.5% or 99%, or 99.5% |
| 98% | 90%, 91%, 92%, 93%, 94%, 95%, 95.5% 96%, 96.5%, |
| | 97%, 97.5%, 98%, 98.5% or 99%, or 99.5% |
| 98.5% | 90%, 91%, 92%, 93%, 94%, 95%, 95.5% 96%, 96.5%, |
| | 97%, 97.5%, 98%, 98.5% or 99%, or 99.5% |
| 99.5% | 90%, 91%, 92%, 93%, 94%, 95%, 95.5% 96%, 96.5%, |
| | 97%, 97.5%, 98%, 98.5% or 99%, or 99.5% |
| 100% | 90%, 91%, 92%, 93%, 94%, 95%, 95.5% 96%, 96.5%, |
| | 97%, 97.5%, 98%, 98.5% or 99%, or 99.5% |

Accordingly, any clustering algorithm known in the art or disclosed elsewhere herein could cluster those organisms sharing common numbers, or percentages, of similar proteins. Subsequently, distances can be calculated based on the number of shared or percent of similar proteins shared between strains. In certain embodiments, distance can be normalized by genome size or other genomic parameters. In other embodiments, the distance can be weighted by amount of difference by combining gene gain/loss with overall sequence divergence. Alternatively, the distance can be weighted by amount of difference by combining gene gain/loss with protein-group specific sequence divergence.

Changing the distance metrics, the clustering parameter, or the clustering algorithm can alter the organisms to which a given organism clusters. For example, organisms that cluster together using the number of shared similar proteins may not cluster together when another clustering parameter is used for classification. In this manner, a database of information can be collected on a given collection of organisms such that visualization of organism clusters can be performed easily and quickly by selecting a given clustering parameter in the database. In certain embodiments, a group identifier, referred to herein as a “pmorph” is assigned to a cluster for a given clustering parameter. The pmorph may incorporate dimensionality, vector, hierarchical, clustering parameter, or other organization implicit in the individual cluster.

In some embodiments, the method can use a known, named organism to anchor the classification. In such a method, the classification would be normalized based on the known organism. In other embodiments, the method disclosed herein can be applied to collections of novel isolates. Before clustering sequences as described herein, the organisms to be classified can be selected, or screened, based on 16S rDNA sequence or other identification methods. In this manner, large collections of organisms can be screened based on coarser classification method in order to select similar organisms for classifying according to the methods disclosed herein.

### IV. Classifying

The methods disclosed herein classify the organisms based on the clusters identified. The method of classification disclosed herein can be applied to a collection of organisms in order to classify organisms based on any given clustering parameter. In certain embodiments, the method comprises selection of a classifying method and/or parameters used. For example, the method can be used on a collection of isolated organisms in order to determine if the collection contains duplications of organisms. Duplicated organisms in a collection would cluster together under any given clustering parameter. Once an organism is classified as a duplicated organism, the collection can be reduced in size by removing the duplicated deposits of the organism and/or by merging all redundant ORFs shared between duplicates into single a single item in the collection. Accordingly, provided herein is a method of reducing the size of a collection of organisms by classifying the collection according to the methods disclosed herein to determine if any organism is duplicated, and removing duplicated deposits of any organism and/or merging the sequence data of these organisms. Collections of organisms can be collections of bacterial or non-bacterial organisms, such as archea or fungal organisms or viruses. The number of organisms in a collection can be about 10, 50, 100, 200, 500, 1000, 10,000, 100,000, 1×10⁶, 1×10⁷, 1×10⁸, ×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, 1×10²⁰ or more organisms.

### V. Computer Systems and Data Storage Devices

The methods for classifying organisms disclosed herein, in whole or in part, may be implemented using a machine, computer system or equivalent, within which a set of instructions for causing the computer or machine to perform any one or more of the protocols or methodologies disclosed herein may be executed. Thus, in certain embodiments, at least one of the steps of the method (e.g., obtaining and/or selecting sequence information; generating a distance metric; clustering; and classifying) is performed using a processor or computer system. The machine may be connected (e.g., networked) to other machines, e.g., in a Local Area Network (LAN), an intranet, an extranet, or the Internet, or any equivalents thereof. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. The term “machine” shall also be taken to include any collection of machines, computers or products of manufacture that individually or jointly execute a set (or multiple sets) of instructions to perform any one or more of the methodologies of the invention.

A computer system for implementing the method, or individual steps of the method, disclosed herein can comprise a processing device (processor), a main memory (e.g., read-only memory (ROM), flash memory, dynamic random access memory (DRAM) such as synchronous DRAM (SDRAM) or Rambus DRAM (RDRAM), etc.), a static memory (e.g., flash memory, static random access memory (SRAM), etc.), and a data storage device, which communicate with each other via a bus.

A processor represents one or more general-purpose processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor may also be one or more special-purpose processing devices such as an application specific integrated circuit (ASIC), a field programmable gate array (FPGA), a digital signal processor (DSP), network processor, or the like. In alternative embodiments the processor is configured to execute the instructions (e.g., processing logic) for performing the operations and steps discussed herein. The computer system further can comprise a network interface device. The computer system also may include a video display unit (e.g., a liquid crystal display (LCD) or a cathode ray tube (CRT)), an alphanumeric input device (e.g., a keyboard), a cursor control device (e.g., a mouse), and a signal generation device (e.g., a speaker).

In some embodiments, the data storage device (e.g., drive unit) comprises a computer-readable storage medium on which is stored one or more sets of instructions (e.g., software) embodying any one or more of the protocols, methodologies or functions of this invention. The instructions may also reside, completely or at least partially, within the main memory and/or within the processor during execution thereof by the computer system, the main memory and the processor also constituting machine-accessible storage media. The instructions may further be transmitted or received over a network via the network interface device. A computer-readable storage medium can be used to store data structure sets that define user identifying states and user preferences that define user profiles. Data structure sets and user profiles may also be stored in other sections of computer system, such as static memory.

While the computer-readable storage medium in an exemplary embodiment is a single medium, the term “machine-accessible storage medium” can be taken to include a single medium or multiple media (e.g., a centralized or distributed database, and/or associated caches and servers) that store the one or more sets of instructions. The term “machine-accessible storage medium” can also be taken to include any medium that is capable of storing, encoding or carrying a set of instructions for execution by the machine and that cause the machine to perform any one or more of the methodologies of the present invention. The term “machine-accessible storage medium” shall accordingly be taken to include, but not be limited to, solid-state memories, and optical and magnetic media.

Non-limiting embodiments include:
1. A method of classifying organisms comprising:
   (a) generating a distance measurement between at least two organisms, wherein the distance measurement is generated by comparing whole or partial genomic DNA sequence data or predicted amino acid sequences from at least 2 organisms;
   (b) clustering said organisms based on the distance measurements; and,
   (c) classifying the organisms based on the clustering in step (b).
2. The method of embodiment 1, further comprising step (d) of isolating at least one organism as classified in step (c).
3. The method of any one of embodiments 1 or 2, wherein the expressed amino acid sequences are predicted by identifying open reading frames (ORFs) in the whole or partial genomic DNA sequence of the at least two organisms, and predicting the expressed amino acid sequences from the identified ORFs.
4. The method of any one of embodimentss 1 - 3, wherein generating a distance measurement comprises comparing the predicted amino acid sequences from the at least two organisms to each other using a distance or a clustering algorithm.
5. The method of any one of embodiments 1 - 4, wherein generating a distance measurement comprises creating based on sequence similarity, physiological characteristics, and/or evolutionary history protein similarity groups.
6. The method of embodiment 5, wherein the protein similarity groups are created using CD-HIT, ITEP, get homologs, or LSBR.
7. The method of any one of embodiments 5 or 6, wherein a gene presence/absence matrix is created based on amino acid sequences predicted to not be present in all compared organisms.
8. The method of any one of embodiments 1-7, wherein the comparison is normalized based on protein-group specific sequence divergence or overall distances.
9. The method of any one of embodiments 1-8, wherein the distance between organisms is calculated based on percent of similar proteins shared between organisms.
10. The method of embodiment 9, wherein similar proteins have amino acid sequences that are at least 98.5% identical.
11. The method of embodiment 9 or 10, wherein distance is normalized by genome size, or distances are weighted based on amount of sequence difference.
12. The method of any of embodiments 1-4, wherein clustering is performed using organism distance matric for hierarchical or high-dimensionality or density-based spatial clustering.
13. The method of embodiment 12, wherein clustering is performed using DB SCAN.
14. The method of embodiment 12, wherein a neighborhood size corresponding to at least 98.5% shared proteins is used.
15. The method of any one of embodiments 1-14, wherein the classifying comprise assigning each cluster a protein morphotype (pmorph).
16. The method of any one of embodiments 1-15, wherein the at least two organisms are selected based on 16s rDNA sequence.
17. The method of any one of embodiments 1- 16, wherein the classified organisms are prokaryotic organisms.
18. The method of any one of embodiments 1-17, wherein the method is performed on a computer.
19. The method of embodiment 17, wherein the at least two organisms are from a collection or organisms, wherein one of the at least two organisms classified as the same organism is removed from the collection such that the size of the collection is reduced.
20. The method of embodiment 19, wherein the collection is obtained from environmental samples.
21. A collection of organisms having been reduced in size by the method of embodiment 19 or 20.

### EXPERIMENTAL

For these examples, predicted protein sequences from Prodigal gene calls of assembled genome sequences of microbial isolates were generated. These amino acid sequences were clustered at cutoffs of 98.5% amino acid identity using the CD-HIT program. A matrix of shared protein clusters above this cutoff were constructed for all pairwise comparisons of isolates, normalized to the average genome size of the pair. This normalized matrix was clustered using the DBSCAN algorithm with a neighborhood size corresponding to 98.5% shared proteins. The preceding steps were implemented in a python script using the numpy, scipy, and sklearn numerical libraries.

### Example 1

### Identification and Classification of Resequenced Strains

For these examples, predicted protein sequences from Prodigal gene calls of assembled genome sequences of microbial isolates were generated. These amino acid sequences were clustered at cutoffs of 98.5% amino acid identity using the CD-HIT program. A matrix of shared protein clusters above this cutoff were constructed for all pairwise comparisons of isolates, normalized to the average genome size of the pair. This normalized matrix was clustered using the DBSCAN algorithm with a neighborhood size corresponding to 98.5% shared proteins. The preceding steps were implemented in a python script using the numpy, scipy, and sklearn numerical libraries.

A test comparing isolates sequenced twice confirmed that classification by pmorphs with the cutoffs described above is consistent despite sequencing, assembly, and protein prediction noise, as no resequensed genome was classified as a different pmorph. These cutoffs correspond to an average of 25 amino acids in a protein and 72 predicted genes.

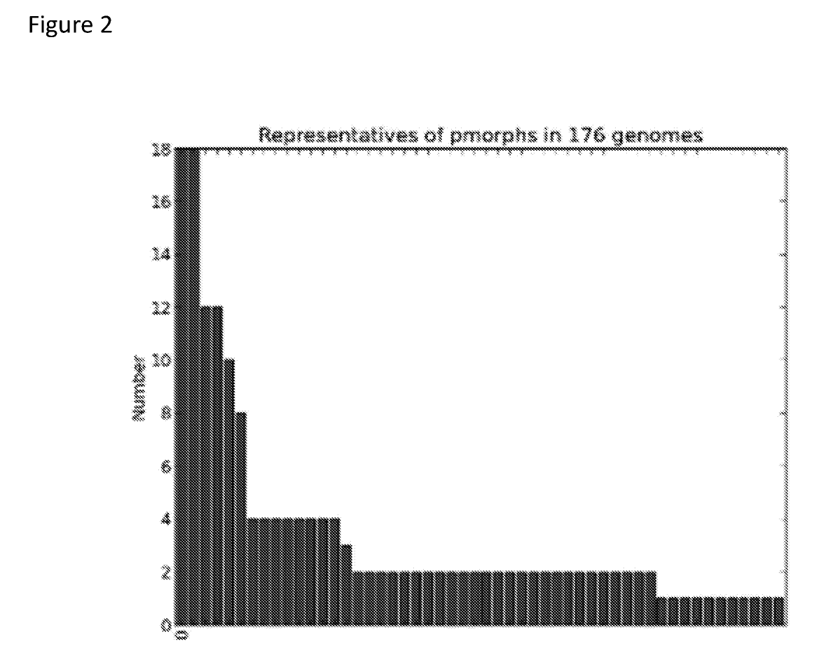

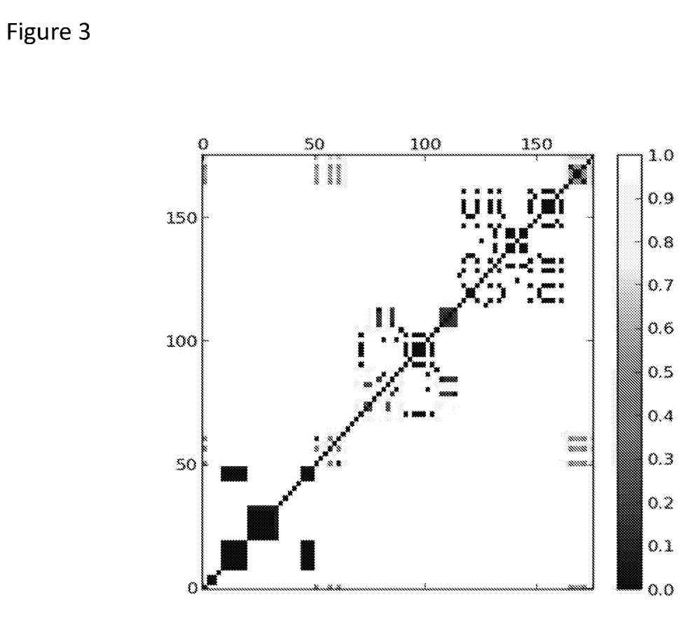

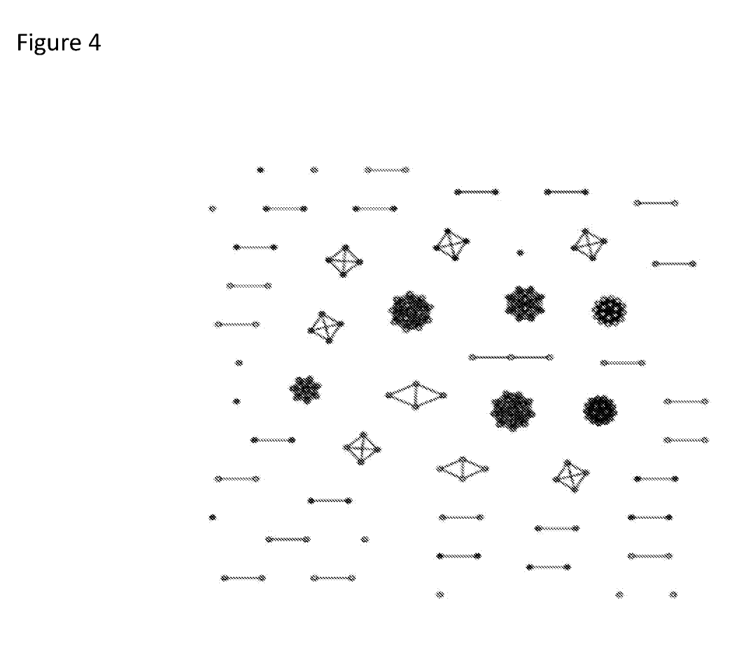

See FIG. 2 for a histogram of duplicated isolates in this test set, FIG. 3 for a heatmap of shared protein cluster distances, and FIG. 4 for a network clustering of isolates: Circles indicate isolate genomes, groups with the same color are clustered together, and distance within group is proportional with number of shared proteins

### Example 2

### Identification and Classification of 5,650 Bacterial Isolates

Below is a pmorph classification using the parameters described above in Example 1 applied to a collection of over 5,650 bacteria, demonstrating its utility for accurate classification of clonal isolates in a large and novel culture collection, and the utility for identifying duplications in a collection. Comparison of the pmorph classification relative to identical rRNA sequences indicates that pmorphs are a higher-resolution classification. There is significant micro-heterogeneity between organisms with identical 16S genes, and great diversity within groups that share a named bacteria with 16S sequence of greatest homology to the isolates.
5,650 unique isolates

1,018 unique rRNAs

101 Genera

272 species
Resulted in:

3,274 distinct pmorph classification groups
0.09% of isolates have more than one pmorph—all isolates classified in different pmorphs had one extremely fragmented or incomplete genome sequence.

Homogeneity: 0.864

Completeness: 1.000
33.89% of identical rRNAs have more than one pmorph

Homogeneity: 0.988

Completeness: 0.756
62.15% of rRNA nearest neighbor identifiers have>one pmorph

Homogeneity: 0.995

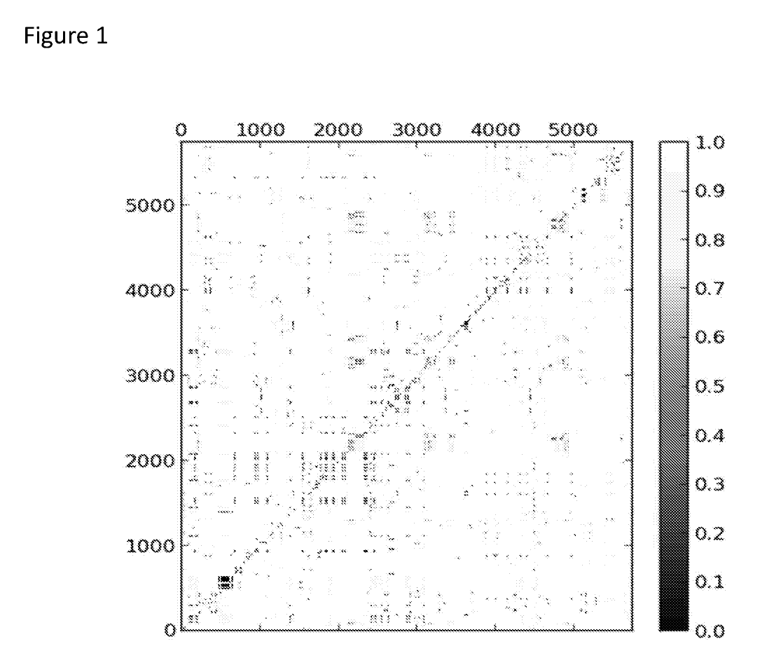

Completeness: 0.546
See FIG. 1 for a heatmap of shared protein cluster distances.

Units, prefixes, and symbols may be denoted in their SI accepted form. Numeric ranges are inclusive of the numbers defining the range.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method of classifying organisms comprising:
(a) generating a distance measurement between at least two organisms, wherein the distance measurement is generated by comparing whole or partial genomic DNA sequence data or predicted amino acid sequences from at least 2 organisms;
(b) clustering said organisms based on the distance measurements; and,
(c) classifying the organisms based on the clustering in step (b).

2. The method of claim 1, further comprising step (d) of isolating at least one organism as classified in step (c).

3. The method of claim 1, wherein the expressed amino acid sequences are predicted by identifying open reading frames (ORFs) in the whole or partial genomic DNA sequence of the at least two organisms, and predicting the expressed amino acid sequences from the identified ORFs.

4. The method of claim 1, wherein generating a distance measurement comprises comparing the predicted amino acid sequences from the at least two organisms to each other using a distance or a clustering algorithm.

5. The method of claim 1, wherein generating a distance measurement comprises creating based on sequence similarity, physiological characteristics, and/or evolutionary history protein similarity groups.

6. The method of claim 5, wherein the protein similarity groups are created using CD-HIT, ITEP, get_homologs, or LSBR.

7. The method of claim 5, wherein a gene presence/absence matrix is created based on amino acid sequences predicted to not be present in all compared organisms.

8. The method of claim 1, wherein the comparison is normalized based on protein-group specific sequence divergence or overall distances.

9. The method of claim 1, wherein the distance between organisms is calculated based on percent of similar proteins shared between organisms.

10. The method of claim 9, wherein similar proteins have amino acid sequences that are at least 98.5% identical.

11. The method of claim 9, wherein distance is normalized by genome size, or distances are weighted based on amount of sequence difference.

12. The method of claim 1, wherein clustering is performed using organism distance matric for hierarchical or high-dimensionality or density-based spatial clustering.

13. The method of claim 12, wherein clustering is performed using DBSCAN.

14. The method of claim 12, wherein a neighborhood size corresponding to at least 98.5% shared proteins is used.

15. The method of claim 1, wherein the classifying comprise assigning each cluster a protein morphotype (pmorph).

16. The method of claim 1, wherein the at least two organisms are selected based on 16s rDNA sequence.

17. The method of claim 1, wherein the classified organisms are prokaryotic organisms.

18. The method of claim 1, wherein the method is performed on a computer.

19. The method of claim 17, wherein the at least two organisms are from a collection or organisms, wherein one of the at least two organisms classified as the same organism is removed from the collection such that the size of the collection is reduced.

20. The method of claim 19, wherein the collection is obtained from environmental samples.

21. A collection of organisms having been reduced in size by the method of claim 19.
